(19) United States
(12) Patent Application Publication
(10) Pub. No.: US 2014/0088425 A1
(43) Pub. Date: Mar. 27, 2014

(54) SINGLE CHIP AND HANDHELD ELECTRONIC DEVICE

(71) Applicant: INDUSTRIAL TECHNOLOGY RESEARCH INSTITUTE, CHUTUNG (TW)

(72) Inventors: Song-Nien Tang, Hsinchu City (TW); Fu-Chiang Jan, Jhuolan Township (TW); Kun-Ta Wu, Nantou City (TW); Guo-Zua WU, Taichung City (TW)

(73) Assignee: INDUSTRIAL TECHNOLOGY RESEARCH INSTITUTE, CHUTUNG (TW)

(21) Appl. No.: 13/720,400

(22) Filed: Dec. 19, 2012

(30) Foreign Application Priority Data
Sep. 27, 2012 (TW) 101135558

(51) Int. Cl. A61B 8/00; A61B 8/08; A61B 8/14

(52) U.S. Cl. CPC A61B 8/4472; A61B 8/14; A61B 8/54; A61B 8/5207; A61B 8/461
USPC 600/443

(57) ABSTRACT

A single chip includes an analog module, an ultrasound imaging module, a wireless network module, a switch circuit and a central processing unit (CPU). The ultrasound imaging module controls an ultrasound front end, and the wireless network module controls a radio-frequency (RF) front end. The CPU controls the switch circuit to electrically connect the analog module to the ultrasound imaging circuit or the wireless network module.

This application claims the benefit of Taiwan application Serial No. 101135558, filed Sep. 27, 2012, the disclosure of which is incorporated by reference herein in its entirety.

### TECHNICAL FIELD

The disclosed embodiments relate a single chip and a handheld electronic device.

### BACKGROUND

An ultrasonic wave (ultrasound) is a mechanic wave generated by a piezoelectric crystal under an effect of an electric field. A sonic wave having a frequency over 20 kHz is regarded as an ultrasound. The ultrasound prevails in applications of examination, measurement and control purposes. For example, the ultrasound is applied for thickness measurement, distance measurement, medical treatments, medical diagnosis and ultrasound imaging (ultrasonography). Alternatively, by processing a material with the ultrasound, certain physical, chemical or biological properties or statuses of the material may be accelerated or changed.

An ultrasound imaging system is extensively implemented for biomedical detections. In ultrasonography, imaging is mainly achieved by pulse-echo. A principle of ultrasonography is summarized as below. A short pulse is transmitted by each array element of a transmitter. With beamforming, a time delay and a gain size of the pulses of each channel are adjusted to focus all the array signals at a position of a fixed depth on a scan line. The signals originally in a digital form are then converted to analog signals by a digital-to-analog converter (DAC) in an analog module, and the electric signals are further converted to ultrasonic signals by a transducer array and transmitted.

At a receiver, the transducer array first converts the mechanic waves into electric signals, and the signals of each channel are amplified, filtered, and sampled by an analog-to-digital converter (ADC) in an analog module. According to each sampling point on the scan line, the time delay and gain size of the signals of each channel are dynamically adjusted, and the signals of all the channels are added. A signal strength after focusing is retrieved. Next, a subsequent beam points to a next scan line, followed by iterating the above imaging process. An image format of an image composed by all the scan lines is converted to a grid, and a final corresponding image is displayed on a display device.

### SUMMARY

The disclosure is directed to a single chip and a handheld electronic device.

According to one embodiment, a single chip is provided. The single chip comprises an analog module, an ultrasound imaging module, a wireless network module, a switch circuit and a central processing unit (CPU). The ultrasound imaging module controls an ultrasound front end, and the wireless network module controls a radio-frequency (RF) front end. The CPU controls the switch circuit to electrically connect the analog module to the ultrasound imaging module or the wireless network module.

According to another embodiment, a handheld electronic device is provided. The handheld electronic device comprises an ultrasound front end, an RF front end, a single chip and a multiplexer. The single chip comprises an analog module, an ultrasound imaging module, a wireless network module, a switch circuit and a CPU. The ultrasound imaging module controls the ultrasound front end, and the wireless network module controls the RF front end. The CPU controls the switch circuit to electrically connect the analog module to the ultrasound imaging module or the wireless network module. The multiplexer selectively couples the ultrasound front end or the RF front end to the analog module.

### BRIEF DESCRIPTION OF THE DRAWINGS

FIG. 1 is a schematic diagram of a computer system.

FIG. 2 is a schematic diagram of a computer system operating under a wireless network operating mode according to a first embodiment.

FIG. 3 is a schematic diagram of a computer system operating under an ultrasound imaging operating mode according to a first embodiment.

FIG. 4 is a schematic diagram of a computer system operating under a wireless network and ultrasound imaging operating mode according to a first embodiment.

FIG. 5 is a schematic diagram of a computer system according to a second embodiment.

FIG. 6 is a schematic diagram of a computer system according to a third embodiment.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

### DETAILED DESCRIPTION

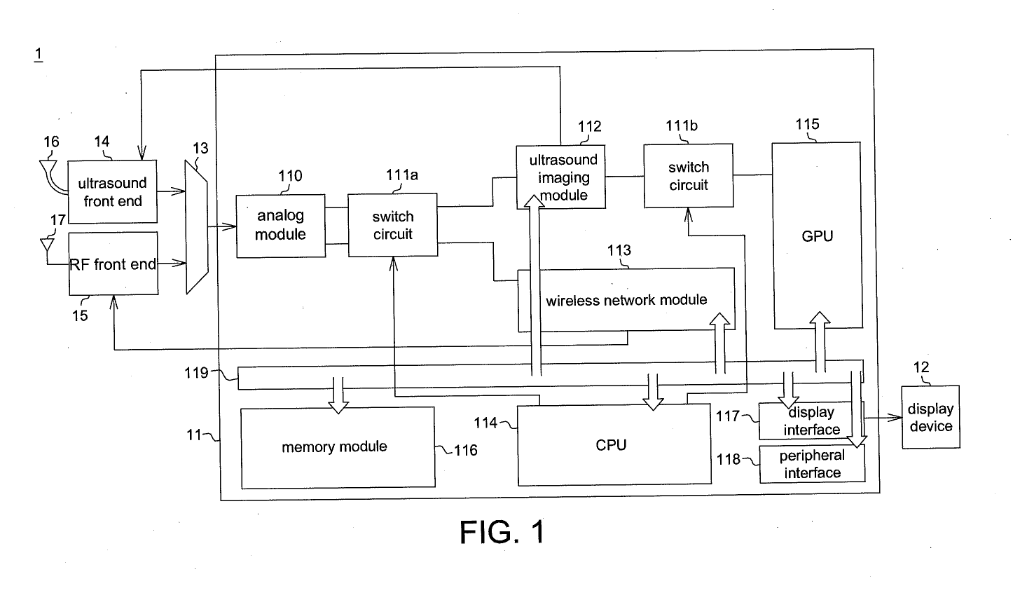

FIG. 1 shows a schematic diagram of a computer system 1 according to one embodiment. For example, the computer system 1 is a handheld electronic device such as a mobile phone or a handheld medical diagnostic apparatus. The computer system 1 comprises a single chip 11, a display device 12, a multiplexer 13, an ultrasound front end 14, a radio-frequency (RF) front end 15, an ultrasound probe 16, and an antenna 17. The ultrasound front end 14 is coupled to the ultrasound probe 16, and the RF front end 15 is coupled to the antenna 17. Under an ultrasound imaging operating mode of the computer system 1, the multiplexer 13 electrically connects the ultrasound front end 14 to the single chip 11. By driving by the ultrasound front end 14 with the ultrasound probe 16, the single chip 11 generates an ultrasonic signal. A reflected ultrasonic signal is inputted to the single chip 11 via the ultrasound front end 14, and a corresponding image is displayed by the display device 12.

The single chip 11 comprises an analog module 110, a switch circuit 111a, a switch circuit 111b, an ultrasound imaging module 112, a wireless network module 113, a CPU 114, a graphics processing unit (GPU) 115, a memory module 116, a display interface 117, a peripheral interface 118 and a bus 119. The bus 119 is coupled to the ultrasound imaging module 112, the wireless network module 113, the CPU 114, the GPU 115, the memory module 116, the display interface 117 and the peripheral interface 118. The peripheral interface 118 couples peripheral devices such as a keyboard or a mouse. The display interface 117 drives the display device 12. The memory module 116 stores data.

The ultrasound imaging module 112 controls the ultrasound front end 14, and the wireless network module 113 controls the RF front end 15. The CPU 114 controls the switch circuit 111a to electrically connect the analog module 110 to the ultrasound imaging module 112 or the wireless network module 113.

### First Embodiment

### Wireless Network Operating Mode

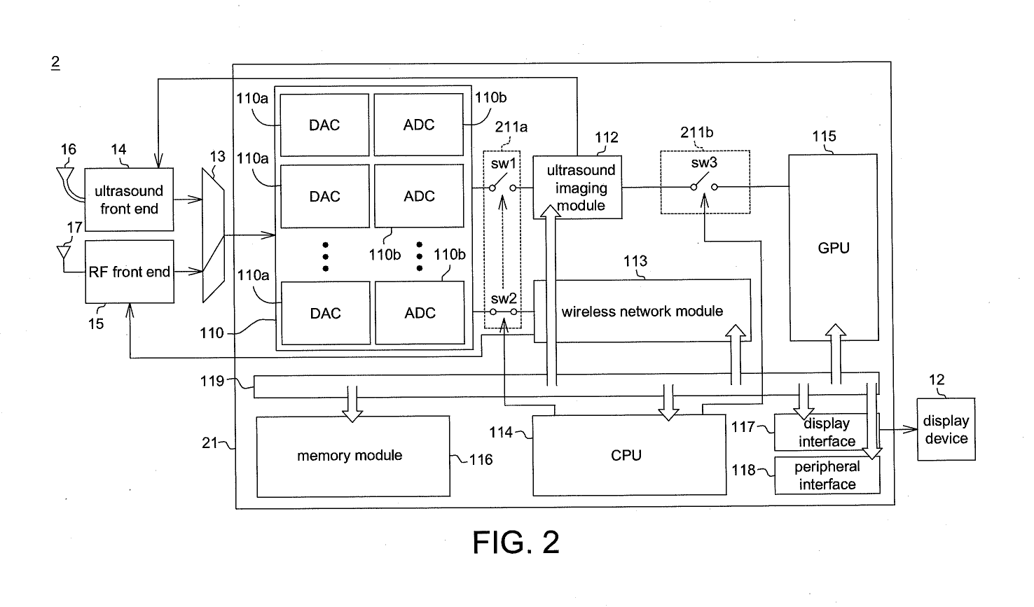

FIG. 2 shows a schematic diagram of a computer system operating under a wireless network operating mode according to a first embodiment. Referring to FIGS. 1 and 2, the computer system 1 in FIG. 1 is exemplified by a computer system 2 depicted in FIG. 2, and the single chip 11 in FIG. 1 is exemplified by a single chip 21 depicted in FIG. 2. Further, the switch circuit 111a in FIG. 1 is exemplified by a switch circuit 211a depicted in FIG. 2, and the switch circuit 111b in FIG. 1 is exemplified by a switch circuit 211b depicted in FIG. 2. The switch circuit 211a comprises a switch SW1 and a switch SW2, and the switch circuit 211b comprises a switch SW3. The analog module 110 comprises digital-to-analog converters (DACs) 110a and analog-to-digital converters (ADCs) 110b. The DACs 110a convert digital signals generated by the ultrasound imaging module 112 to analog signals and output the analog signals to the ultrasound front end 14, or convert digital signals generated by the wireless network module 113 to analog signals and output the analog signals to the RF front end 15. The ADCs 110b convert analog signals generated by the ultrasound font end 14 to digital signals and output the digital signals to the ultrasound imaging module 112, or convert analog signals generated by the RF front end 15 to digital signals and output the digital signals to the wireless network module 113.

The computer system 2 can be operated under a wireless network mode to utilize the wireless network module 113. Under the wireless network operating module, the CPU 114 controls the switch SW2 of the switch circuit 211a to electrically connect the DACs 110a and the ADCs 110b of the analog module 110 to the wireless network module 113, and not to electrically connect the DACs 110a and the ADCs 110b of the analog module 110 to the ultrasound imaging module 112. Under the wireless network operating mode, the CPU 114 further controls the switch SW3 of the switch circuit 211b not to electrically connect the ultrasound imaging module 112 to the GPU 115. In other words, the CPU 114 allots the DACs 110a and the ADCs 110b of the analog module 110 for the use of the wireless network module 113.

Since the computer system 2 does not employ the ultrasound imaging module 112 under the wireless network operating mode, the CPU 114 is able to further control a power management module to stop powering the ultrasound imaging module 112 under the wireless network operating mode, thereby reducing unnecessary power consumption.

### Ultrasound Imaging Operating Mode

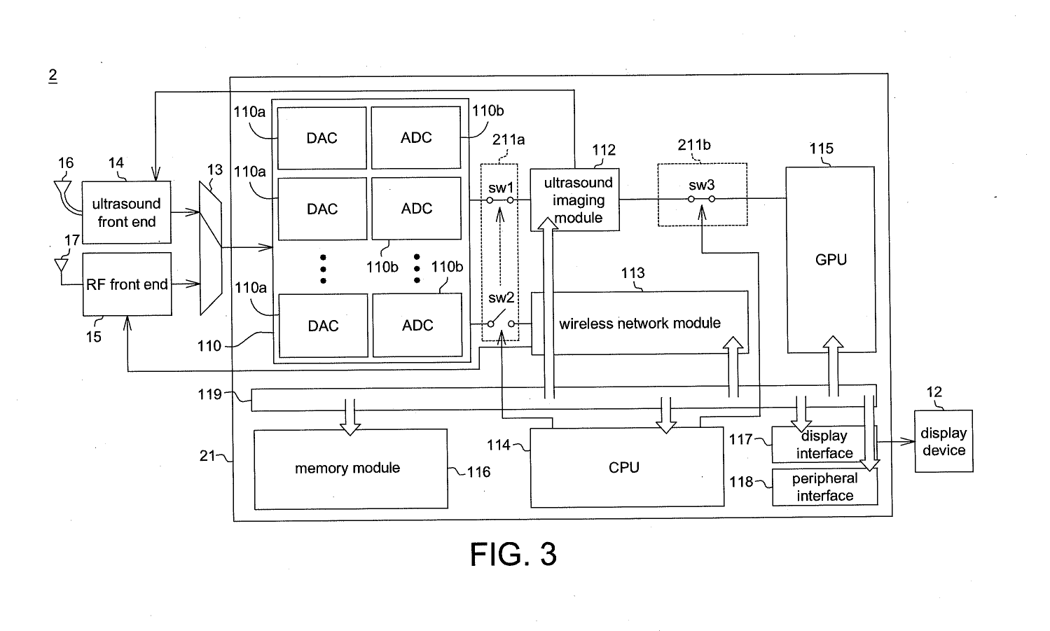

FIG. 3 shows a schematic diagram of a computer system operating under an ultrasound imaging operating module according to the first embodiment. The computer system 2 can be operated under the ultrasound imaging operating mode to utilize the ultrasound imaging module 112. Under the ultrasound imaging operating mode, the CPU 114 controls the switch SW1 of the switch circuit 211a to electrically connect the DACs 110a and the ADCs 110b of the analog module 110 to the ultrasound imaging module 112, and not to electrically connect the DACs 110a and the ADCs 110b of the analog module 110 to the wireless network module 113. In other words, the CPU 114 allots the DACs 110a and the ADCs 110b of the analog module 110 for the use of the ultrasound imaging module 112.

The ultrasound imaging module 112 performs an ultrasound imaging computation, e.g., a digital beamforming (DBF) algorithm or a Doppler blood flow estimation. To reduce a computation amount of the ultrasound imaging module 112, the CPU 114 further controls the switch SW3 of the switch circuit 211b to electrically connect the ultrasound imaging module 112 to the GPU 115 under the ultrasound imaging operating mode. The GPU 115 further supports the ultrasound imaging computation of the ultrasound imaging module 112.

Since the computer system 2 does not employ the wireless network module 113 under the ultrasound imaging operating mode, the CPU 114 is able to further control a power management module to stop powering the wireless network module 113 under the ultrasound imaging operating mode, thereby reducing unnecessary power consumption.

### Wireless Network and Ultrasound Imaging Operating Mode

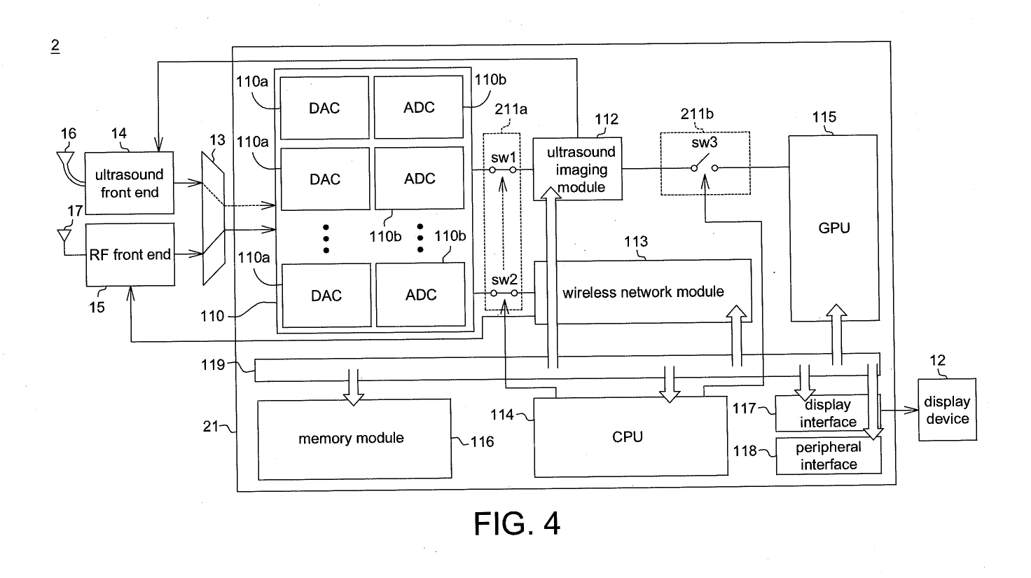

FIG. 4 shows a schematic diagram of a computing system operating under a wireless network and ultrasound imaging operating mode according to the first embodiment. The computer system 2 can be operated under the wireless network and ultrasound imaging operating mode to utilize both the wireless network module 113 and the ultrasound imaging module 112. Under the wireless network and ultrasound imaging operating mode, the CPU 114 controls the switch SW1 of the switch circuit 211a to electrically connect an M number of DACs 110a and an M number of ADCs 110b of the analog module 110 to the ultrasound imaging module 112, and to electrically connect an N number of DACs 110a and an N number of ADCs 110b of the analog module 110 to the wireless network module 113. M and N are non-zero positive integers. Under the wireless network and ultrasound imaging operating mode, the CPU 114 controls the switch SW3 of the switch circuit 211b to electrically connect the ultrasound imaging module 112 to the GPU 115. In other words, the CPU 114 allots the DACs 110a and the ADCs 110b of the analog module 110 for the use of the ultrasound imaging module 112 and the wireless network module 113.

According to a user command or a wireless network signal quality, the CPU 114 may control the switch SW1 of the switch circuit 211a to electrically connect the M number of DACs 110a and the M number of ADCs 110b of the analog module 110 to the ultrasound imaging module 112, and to electrically connect the N number of DACs 110a and the N number of ADCs 110b of the analog module 110 to the wireless network module 113. Through a user interface, a user may input the user command to allot the DACs 110a and the ADCs 110b for the use of the ultrasound imaging module 112 and the wireless network module 113. Alternatively, the CPU 114 first determines the N number of DACs 110a and the N number of ADCs 110b to be used by the wireless network module 113 according to the wireless network signal quality, and then allots the remaining M number of DACs 110a and the M number of ADCs 110b to the ultrasound imaging module 112.

Further, the computer system 2 may also store an ultrasound image generated by the ultrasound imaging module 112 to the memory module 116, and upload the ultrasound image to a medical diagnostic center in real-time via the wireless network module 113. Thus, the medical diagnostic center is allowed to perform diagnosis according to the received ultrasound image.

### Computer Operating Mode

In addition to the above three operating modes, the computer system 2 can also be operated in a computer operating mode. Under the computer operating mode, the CPU 114 turns off the switches SW1, SW2 and SW3. The DACs 110a and the ADCs 110b are electrically connected to neither the wireless network module 113 nor the ultrasound imaging module 112. That is, the computer system 2 is utilized as a common computer. Thus, the CPU 114 is able to further control a power management module to stop powering the analog module 110, the ultrasound imaging module 112 and the wireless network module 113 under the computer operating module, thereby reducing unnecessary power consumption.

### Second Embodiment

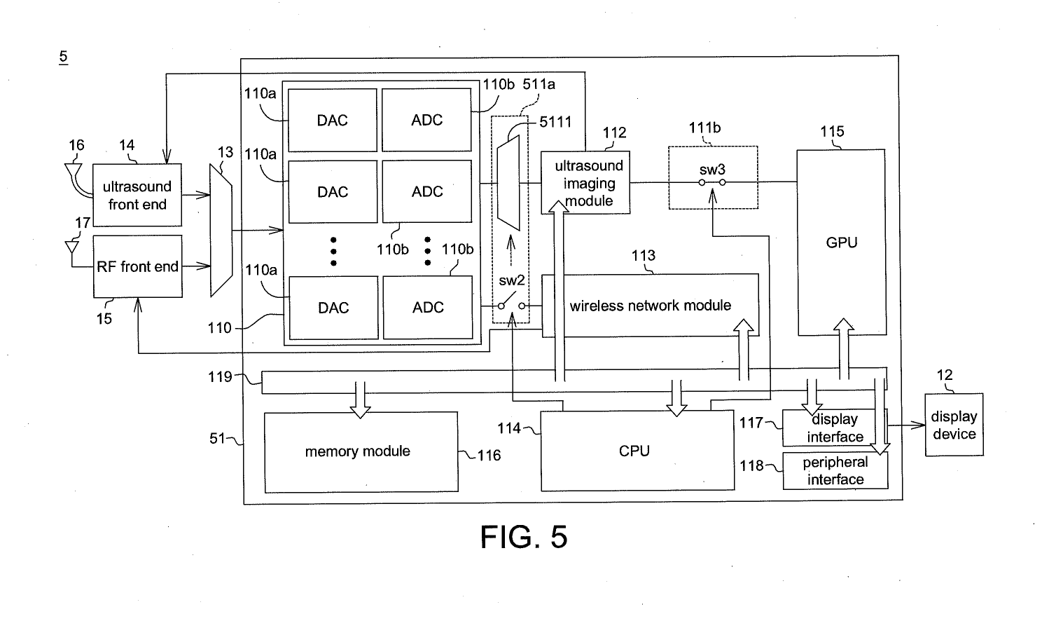

FIG. 5 shows a schematic diagram of a computer system according to a second embodiment. Referring to FIGS. 1, 2 and 5, the computer system 1 in FIG. 1 is exemplified by a computer system 5 depicted in FIG. 5, and the single chip 11 in FIG. 1 is exemplified by a single chip 51 depicted in FIG. 5. Further, the switch circuit 111a in FIG. 1 is exemplified by a switch circuit 511a depicted in FIG. 5. A main difference between the computer system 5 and the computer system 2 is that, the switch circuit 511a comprises an analog multiplexer 5111 and a switch SW2. The analog multiplexer 5111 electrically connects the analog module 110 to the ultrasound imaging module 112, and the switch SW2 electrically connects the analog module 110 to the wireless network module 113.

The CPU 114 adjusts a switching frequency of the analog multiplexer 5111 according to the number of channels of the ultrasound probe 16. When the number of channels of the ultrasound probe 16 is greater than the number of DACs or ADCs, the CPU 114 increases the expandability of the ultrasound computation by increasing the switching frequency of the analog multiplexer 5111. For example, assume that the analog module 110 has eight DACs 110a and eight ADCs 110b. When the number of channels of the ultrasound probe 16 is eight, the CPU 114 performs switching according to an original switching frequency through controlling the analog multiplexer 5111, so that the ultrasound imaging module 112 receives or outputs digital signal corresponding to the eight channels. When the number of channels of the ultrasound probe 16 is changed to sixteen, the CPU 114 doubles the original switching frequency through controlling the analog multiplexer 5111, so that the ultrasound imaging module 112 receives or outputs digital signals corresponding to the sixteen channels.

### Third Embodiment

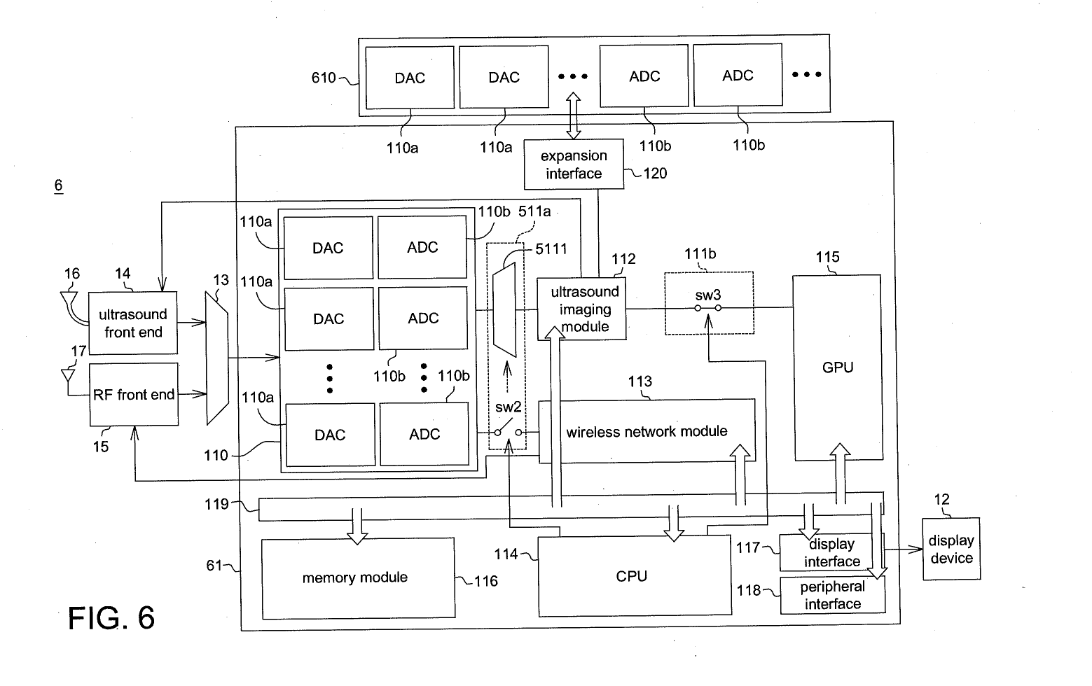

FIG. 6 shows a schematic diagram of a computer system according to a third embodiment. Referring to FIGS. 1, 2 and 6, the computer system 1 in FIG. 1 is exemplified by a computer system 6 depicted in FIG. 6, and the single chip 11 in FIG. 1 is exemplified by a single chip 61 depicted in FIG. 6. A main difference between the single chip 61 and the single chip 51 is that, the single chip 61 further comprises an expansion interface 120, e.g., a Low-Voltage Differential Signaling (LVDS) interface. The expansion interface 120 connects to an external analog module 610, and electrically connects the analog module 610 to the ultrasound imaging module 112. The analog module 610 comprises DACs 110a and ADCs 110b.

Similarly, the DACs 110a of the analog module 610 convert digital signals generated by the ultrasound imaging module 112 to analog signals and output the analog signals to the ultrasound front end 14, or convert digital signals generated by the wireless network module 113 to analog signals and output the analog signal to the RF front end 15. The ADCs 110b of the analog module 610 convert analog signal generated by the ultrasound front end 14 to digital signals and output the digital signals to the ultrasound imaging module 112, or convert analog signal generated by the RF front end 15 to digital signals and output the digital signals to the wireless network module 113.

The single chip 61 further enhances the expansibility of the ultrasound computation via the expansion interface 120. For example, assume that the analog module 610 has eight digital DACs 110a and eight ADCs 110b. When the number of channels of the ultrasound probe 16 is sixteen, the single chip 61 increases the numbers of the DACs 110a and the ADCs 110b by externally connecting to the analog module 610 via the expansion interface 120.

Moreover, assume that the number of channels of the ultrasound probe 16 is 64, and the ultrasound imaging module 112 is capable of processing only digital signals of 32 channels. The GPU 115 may then support the ultrasound imaging module 112 to process digital signals of the remaining 32 channels.

With the descriptions of the embodiments, it is demonstrated that the single chip disclosed in the foregoing embodiments integrates an ultrasound imaging function to a computer single chip, thereby allowing a user to utilize the ultrasound imaging function, network function or computer function through the single chip. Further, as the single chip supports the ultrasound imaging function, developments of handheld electronic devices having an ultrasound examination function are further promoted.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A single chip, comprising:
a first analog module;
an ultrasound imaging module, for controlling an ultrasound front end;
a wireless network module, for controlling a radio-frequency (RF) front end;
a first switch circuit; and
a central processing unit (CPU), for controlling the first switch circuit to electrically connect the first analog module to the ultrasound imaging module or the wireless network module.

2. The single chip according to claim 1, wherein the first switch circuit comprises:
a first switch, for electrically connecting the first analog module to the ultrasound imaging module; and
a second switch, for electrically connecting the first analog module to the wireless network module.

3. The single chip according to claim 1, wherein the first switch circuit comprises:
an analog multiplexer, for electrically connecting the first analog module to the ultrasound imaging module; and
a switch, for electrically connecting the first analog module to the wireless network module.

4. The single chip according to claim 3, wherein the CPU adjusts a switching frequency of the analog multiplexer according to a number of channels of an ultrasonic probe.

5. The single chip according to claim 1, further comprising:
a graphics processing unit (GPU), for supporting a ultrasound imaging computation of the ultrasound imaging module; and
a second switch circuit, controlled by the CPU to electrically connect the ultrasound imaging module to the GPU.

6. The single chip according to claim 5, wherein the second switch circuit is a switch.

7. The single chip according to claim 5, further comprising:
a memory module;
a display interface;
a peripheral interface; and
a bus, coupling the ultrasound imaging module, the wireless network module, the GPU, the memory module, the display interface and the peripheral interface.

8. The single chip according to claim 1, further comprising:
an expansion interface, for externally connecting to a second analog module and electrically connecting the second analog module to the ultrasound imaging module.

9. The single chip according to claim 1, wherein under a wireless network operating mode, the CPU controls the first switch circuit to electrically connect the first analog module to the wireless network module and the not to electrically connect the first analog module to the ultrasound imaging module.

10. The single chip according to claim 1, wherein under an ultrasound imaging operating mode, the CPU controls the first switch circuit to electrically connect the first analog module to the ultrasound imaging module and not to electrically connect the first analog module to the wireless network module.

11. The single chip according to claim 1, wherein under a wireless network and ultrasound imaging operating mode, the CPU controls the first switch circuit to electrically connect the first analog module to the ultrasound imaging module and to electrically connect the first analog module to the wireless network module.

12. The single chip according to claim 11, wherein the first analog module comprises an M number of digital-to-analog converters (DACs), an N number of DACs, an M number of analog-to-digital converters (ADCs) and an N number of ADCs; under the wireless network and ultrasound imaging mode, the CPU controls the first switch circuit to electrically connect the M number of DACs and the M number of ADCs to the ultrasound imaging module and to electrically connect the N number of DACs and the N number of ADCs to the wireless network module according to a user command.

13. The single chip according to claim 11, wherein the first analog module comprises an M number of DACs, an N number of DACs, an M number of ADCs and an N number of ADCs; under the wireless network and ultrasound imaging operating mode, the CPU controls the first switch circuit to electrically connect the M number of DACs and the M number of ADCs to the ultrasound imaging module and to electrically connect the N number of DACs and the N number of ADCs to the wireless network module according to a wireless network signal quality.

14. The single chip according to claim 1, wherein the CPU controls a power management module to stop powering the ultrasound imaging module under a wireless network operating mode.

15. The single chip according to claim 1, wherein the CPU controls a power management module to stop powering the wireless network module under an ultrasound imaging operating mode.

16. The single chip according to claim 1, wherein the CPU controls a power management module to stop powering the ultrasound imaging module, the wireless network module and the first analog module under a computer operating mode.

17. A handheld electronic device, comprising:
an ultrasound front end;
an RF front end;
a single chip, comprising:
a first analog module;
an ultrasound imaging module, for controlling the ultrasound front end;
a wireless network module, for controlling the RF front end;
a first switch circuit; and
a CPU, for controlling the first switch circuit to electrically connect the first analog module to the ultrasound imaging module or the wireless network module
a multiplexer, for selectively electrically connecting the ultrasound front end or the RF front end to the first analog module.

18. The handheld electronic device according to claim 17, wherein the first switch circuit comprises:
a first switch, for electrically connecting the first analog module to the ultrasound imaging module; and
a second switch, for electrically connecting the first analog module to the wireless network module.

19. The handheld electronic device according to claim 17, wherein the first switch circuit comprises:
an analog multiplexer, for electrically connecting the first analog module to the ultrasound imaging module; and
a switch, for electrically connecting the first analog module to the wireless network module.

20. The handheld electronic device according to claim 17, wherein the single chip further comprises:
a GPU, for supporting an ultrasound imaging computation of the ultrasound imaging module; and
a second switch circuit, controlled by the CPU to electrically connect the ultrasound imaging module to the GPU.
